SEQUENCE LISTING (7 sequences)

SEQ ID NO: 1 - DNA, 24 bp, Artificial Sequence
  primer
ccactctgaagaaggaaacactgc

SEQ ID NO: 2 - DNA, 22 bp, Artificial Sequence
  primer
ccagttcttccgattccagcac

SEQ ID NO: 3 - protein, 13 aa, Artificial Sequence
  chemically synthesized substrate
MTSRFRLPAGRTC

SEQ ID NO: 4 - protein, 14 aa, Artificial Sequence
  chemically synthesized substrate
CEGFVKPLTTSTNK

SEQ ID NO: 5 - protein, 10 aa, Artificial Sequence
  PHOSPHORYLATION (4)...(4): chemically synthesized substrate
EPQYEEIPIA

SEQ ID NO: 6 - protein, 9 aa, Artificial Sequence
  chemically synthesized substrate
KLSSIESDV

SEQ ID NO: 7 - protein, 14 aa, Artificial Sequence
  chemically synthesized substrate
CEGFVKPLTTSTNK

(19) United States
(12) Patent Application Publication
(10) Pub. No.: US 2005/0266509 A1
(43) Pub. Date: Dec. 1, 2005

(54) Interaction of NMDA receptor with protein tyrosine phosphatase

(75) Inventors: Jasna Jerecic, San Francisco, CA (US); Roman Urfer, Belmont, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: AGY Therapeutics, Inc.

(21) Appl. No.: 10/913,631

(22) Filed: Aug. 6, 2004

Related U.S. Application Data
(63) Continuation-in-part of application No. 10/910,159, filed on Aug. 2, 2004.
(60) Provisional application No. 60/491,918, filed on Aug. 1, 2003.

(51) Int. Cl. C12Q 1/48

(52) U.S. Cl. 435/15

(57) ABSTRACT

The present invention relates to the identification of a binding between NMDA receptor (NMDA-R) subunits and the protein tyrosine phosphatase PTPMEG. The present invention provides methods for screening a PTP agonist or antagonist that modulates NMDA-R signaling. The present invention also provides methods and compositions for treatment of disorders mediated by abnormal NMDA-R signaling.

### BACKGROUND OF THE INVENTION

In the majority of mammalian excitatory synapses, glutamate (Glu) mediates rapid chemical neurotransmission by binding to three distinct types of glutamate receptors on the surfaces of brain neurons. Although cellular responses mediated by glutamate receptors are normally triggered by exactly the same excitatory amino acid (EAA) neurotransmitters in the brain (e.g., glutamate or aspartate), the different subtypes of glutamate receptors have different patterns of distribution in the brain, and mediate different cellular signal transduction events. One major class of glutamate receptors is referred to as N-methyl-D-aspartate receptors (NMDA-Rs), since they bind preferentially to N-methyl-D-aspartate (NMDA). NMDA is a chemical analog of aspartic acid; it normally does not occur in nature, and NMDA is not present in the brain. When molecules of NMDA contact neurons having NMDA-Rs, they strongly activate the NMDA-R (i.e., they act as a powerful receptor agonist), causing the same type of neuronal excitation that glutamate does. It has been known that excessive activation of NMDA-R plays a major role in a number of important central nervous system (CNS) disorders, while hypoactivity of NMDA-R has been implicated in several psychiatric diseases.

NMDA-Rs contain an NR1 subunit and at least one of four different NR2 and NR3 subunits (designated as NR2A, NR2B, NR2C, and NR2D, NR3A and NR3B). NMDA-Rs are “ionotropic” receptors since they flux ions, such as Ca2+. These ion channels allow ions to flow into a neuron upon depolarization of the postsynaptic membrane, when the receptor is activated by glutamate, aspartate, or an agonist drug.

Protein tyrosine phosphorylation plays an important role in regulating diverse cellular processes. The regulation of protein tyrosine phosphorylation is mediated by the reciprocal actions of protein tyrosine kinases (PTKs) and protein tyrosine phosphatases (PTPs). NMDA-Rs are regulated by protein tyrosine kinases and phosphatases. Phosphorylation of NMDA-R by protein tyrosine kinases results in enhanced NMDA-R responsiveness in neurons (Wang et al., Nature 369:233-235, 1994). NR2B and NR2A have been shown to be the main sites of phosphorylation by protein tyrosine kinases. Protein tyrosine phosphatases, on the other hand, exert opposing effects on the responsiveness of NMDA-R in the neurons (Wang et al, Proc. Natl. Acad. Sci. U.S.A. U.S.A. 93:1721-1725, 1996). It is believed that members of the Src family of protein tyrosine kinases mediate the NMDA-R tyrosine phosphorylation. On the other hand, the identity of the enzyme responsible for the counter dephosphorylation of NMDA-R has been elusive.

### SUMMARY OF THE INVENTION

Methods are provided for identifying a modulator of N-methyl-D-aspartate receptor (NMDA-R) signaling by detecting the ability of an agent to modulate the phosphatase activity of PTPMEG, e.g. on a NMDA-R substrate, on a kinase in a signaling pathway associated with NMDA-R, etc., or to modulate the binding of PTPMEG to NMDA-R. In one embodiment, the modulator is identified by detecting its ability to modulate the phosphatase activity of PTPMEG. In another embodiment, the modulator is identified by detecting its ability to modulate the binding of PTPMEG and the NMDA-R. In another embodiment, methods are provided for identifying a nucleic acid molecule encoding polypeptides that modulate NMDA-R signaling. It is found that active PTPMEG downregulates NMDA-R activity, and inhibitors of PTPMEG can increase the activity of NMDA-R when PTPMEG is present.

In another embodiment of the invention, methods are provided for identifying a modulator of Src protein tyrosine kinase by detecting the ability of an agent to modulate the phosphatase activity of PTPMEG on a Src or on a Src substrate. PTPMEG acts to inactivate Src. Inhibitors of PTPMEG increase Src activity when PTPMEG is present; and activators of PTPMEG decrease Src activity when PTPMEG is present.

Methods are provided for treating a disease associated with abnormal NMDA-R-signaling by administering a modulator of a PTPMEG activity, which directly or indirectly modulates the tyrosine phosphorylation level of the NMDA-R. The modulator may affect the ability of PTPMEG to dephosphorylate NMDA-R, to dephosphorylate kinases in a signaling pathway associated with NMDA-R, and/or the ability of PTPMEG to bind to NMDA-R. In certain embodiments, the modulator is a PTPMEG agonist and the disease to be treated is mediated by excessive NMDA-R signaling. In other embodiments, the modulator is a PTPMEG antagonist and the disease to be treated is mediated by NMDA-R hypofunction.

### BRIEF DESCRIPTION OF THE DRAWINGS

FIG. 1 provides in situ hybridization for PTPMEG in brain sections. It can be seen that the mRNA is expressed in hippocampus, thalamus and cortex.

FIGS. 2A and 2B show that PTPMEG coimmunoprecipitates with NMDA-R from brain tissue.

FIG. 3. Analysis of NR2B phosphorylation using anti NR2B antibody as loading control and NR2B-PY1472 for detection of NR2B tyrosine phosphorylation by Src kinase. Anti-PTPMEG antibody was used for detection of PTPMEG expression. Lane 1: shows NR2B phosphorylation in presence of constitutive active Src kinase. Lane 2: shows decreased NR2B phosphorylation in presence of active PTPMEG (wt). Lane 3: shows NR2B phosphorylation in presence of inactive PTPMEG (cs). Lane 4: untransfected cells show low phosphorylation levels of NR2B in absence of active Src kinase.

FIG. 4. Analysis of Src kinase phosphorylation using Src specific antibodies (pan Src, PY418, PY529). Lane 1: Src phosphorylation in absence of PTPMEG detected by anti-PY 418 and PY-529 antibody. Lane 2: Src is dephosphorylated specifically at its catalytic site at position 418 by PTPMEG (wt). Lane 3: Src phosphorylation at position 418 is unaffected in presence of inactive PTPMEG(cs). Lane 4: untransfected cells show low levels of endogenously active Src, phosphorylated at position 418. Anti-Src antibody was used as loading control for transfected cells.

FIG. 5 depicts the inhibition of PTPMEG by dephostatin, which restores phosphorylation of Src at position 418.

FIG. 6 depicts a gel showing the dephosporylation of protein kinases Src and fyn by PTPMEG.

FIGS. 7A and 7B show the immunoprecipitation of PTPMEG from selected brain tissues.

FIG. 8 depicts an immunohistochemical staining of a hippocampal section with a PTPMEG specific antibody.

### DETAILED DESCRIPTION OF THE EMBODIMENTS

The present invention relates to the discovery that PTPMEG downregulates activity of NMDA-R. It is also found that PTPMEG inactivates Src kinase. In accordance with the discovery, the present invention provides methods for identifying agonists and antagonists of PTPMEG that modulate NMDA-R signaling, and for treating conditions mediated by abnormal NMDA-R signaling. The following description provides guidance for making and using the compositions of the invention, and for carrying out the methods of the invention.

### DEFINITIONS

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). Although any methods and materials similar or equivalent to those described herein can be used in the practice or testing of the present invention, the preferred methods and materials are described. The following definitions are provided to assist the reader in the practice of the invention.

As used herein, the term “acute insult to the central nervous system” includes short-term events that pose a substantial threat of neuronal damage mediated by glutamate excitotoxicity. These include ischemic events (which involve inadequate blood flow, such as a stroke or cardiac arrest), hypoxic events (involving inadequate oxygen supply, such as drowning, suffocation, or carbon monoxide poisoning), trauma to the brain or spinal cord (in the form of mechanical or similar injury), certain types of food poisoning which involve an excitotoxic poison such as domoic acid, and seizure-mediated neuronal degeneration, which includes certain types of severe epileptic seizures. It can also include trauma that occurs to another part of the body, if that trauma leads to sufficient blood loss to jeopardize blood flow to the brain (for example, as might occur following a shooting, stabbing, or automobile accident).

The term “agent” includes any substance, molecule, element, compound, entity, or a combination thereof. It includes, but is not limited to, e.g., protein, oligopeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

As used herein, an “agonist” or “activator” is a molecule which, when interacting with (e.g., binding to) a target protein (e.g., PTPMEG, NMDA-R), increases or prolongs the amount or duration of the effect of the biological activity of the target protein. By contrast, the term “antagonist,” or “inhibitor” as used herein, refers to a molecule which, when interacting with (e.g., binding to) a target protein, decreases the amount or the duration of the effect of the biological activity of the target protein. Agonists and antagonists may include proteins, nucleic acids, carbohydrates, antibodies, or any other molecules that decrease the effect of a protein.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

The term “biological preparation” refers to biological samples taken in vivo and in vitro (either with or without subsequent manipulation), as well as those prepared synthetically. Representative examples of biological preparations include cells, tissues, solutions and bodily fluids, a lysate of natural or recombinant cells.

As used herein, the term “functional derivative” of a native protein or a polypeptide is used to define biologically active amino acid sequence variants that possess the biological activities (either functional or structural) that are substantially similar to those of the reference protein or polypeptide. Thus, a functional derivative of PTPMEG will retain, among other activities, the ability to bind to, and dephosphorylate Src, and to bind to NMDA-R.

NMDA receptors are a subclass of excitatory, ionotropic L-glutamate neurotransmitter receptors. They are heteromeric, integral membrane proteins being formed by the assembly of the obligatory NR1 subunit together with modulatory NR2 subunits. The NRI subunit is the glycine binding subunit and exists as 8 splice variants of a single gene. The glutamate binding subunit is the NR2 subunit, which is generated as the product of four distinct genes, and provides most of the structural basis for heterogeneity in NMDA receptors. In the hippocampus and cerebral cortex, the active subunit NMDAR1 is associated with 1 of 2 regulatory epsilon subunits: NMDAR2A or NMDAR2B and NR3. Unless otherwise specified, the term “NMDA-R” or “NMDA receptor” as used herein refers to an NMDA receptor molecule that has an NR1 subunit and at least one NR2A or NR2B subunit.

An exemplary NR1 subunit is the human NMDAR1 polypeptide. The sequence of the polypeptide and corresponding nucleic acid may be obtained at Genbank, accession number L05666, and is published in Planells-Cases et al. (1993) P.N.A.S. 90(11):5057-5061. An exemplary NR2 subunit is the human NMDAR2A polypeptide. The sequence of the polypeptide and corresponding nucleic acid may be obtained at Genbank, accession number U09002, and is published in Foldes et al. (1994) Biochim. Biophys. Acta 1223 (1):155-159. Another NR2 subunit is the human NMDAR2B polypeptide. The sequence of the polypeptide and corresponding nucleic acid may be obtained at Genbank, accession number U11287, and is published in Adams et al. (1995) Biochim. Biophys. Acta 1260 (1):105-108.

PTPMEG refers to a protein tyrosine phosphatase, also known as PTPN3. An exemplary PTPMEG molecule is the human polypeptide. The sequence of the polypeptide and corresponding nucleic acid may be obtained at Genbank, accession number NM_{—}002830.

A fundamental process for regulating the function of NMDA receptors and other ion channels in neurons is tyrosine phosphorylation. A phosphatase enzyme may act on NMDA-R directly, to dephosphorylate one or more of the NMDA-R subunits. Alternatively a phosphatase enzyme may act on NMDA-R indirectly, by dephosphorylating a protein tyrosine kinase (PTK) in a signaling pathway. For example, a phosphatase that acts to decrease the activity of a PTK that phosphorylates NMDA-R, will indirectly result in decreased phosphorylation of NMDA-R.

PTKs can potentiate the function of recombinant NMDA receptors. The family of Src kinases comprises a total of nine members, five of which Src, Fyn, Lyn, Lck, and Yes are known to be expressed in the CNS. All members of the Src family contain highly homologous regions the C-terminal, catalytic, Src homology 2, and Src homology 3 domains. The kinase activity of Src protein is normally inactivated by phosphorylation of the tyrosine residue at position 527, which is six residues from the C-terminus. Hydrolysis of phosphotyrosine 527 by a phosphatase enzyme normally activates c-Src. Active Src is also phosphorylated at Y-418. Dephosphorylation at this residue by PTPMEG is found to inactivate Src.

As used herein, the term “NMDA-R signaling” refers to signal-transducing activities in the central nervous system that are involved in the various cellular processes such as neurodevelopment, neuroplasticity, and excitotoxicity. NMDA-R signaling affects a variety of processes including, but not limited to, neuron migration, neuron survival, synaptic maturation, learning and memory, and neurodegeneration.

The term “NMDA-R hypofunction” is used herein to refer to abnormally low levels of signaling activity of NMDA-Rs on CNS neurons. For example, NMDA-R hypofunction may be caused by abnormally low phosphotyrosine level of NMDA-R. NMDA-R hypofunction can occur as a drug-induced phenomenon. It can also occur as an endogenous disease process.

The term “modulation” as used herein refers to both upregulation, (i.e., activation or stimulation), for example by agonizing; and downregulation (i.e. inhibition or suppression), for example by antagonizing, of a bioactivity. As used herein, the term “modulator of NMDA-R signaling” refers to an agent that is able to alter an NMDA-R activity that is involved in the NMDA-R signaling pathways. Modulators include, but are not limited to, both “activators” and “inhibitors” of NMDA-R tyrosine phosphorylation. An “activator” is a substance that directly or indirectly enhances the tyrosine phosphorylation level of NMDA-R, and thereby causes the NMDA receptor to become more active. The mode of action of the activator may be direct, e.g., through binding the receptor, or indirect, e.g., through binding another molecule which otherwise interacts with NMDA-R (e.g., PTPMEG, Src, Fyn, etc). Conversely, an “inhibitor” directly or indirectly decreases the tyrosine phosphorylation of NMDA-R, and thereby causes NMDA receptor to become less active. The reduction may be complete or partial. As used herein, modulators of NMDA-R signaling encompass PTPMEG antagonists and agonists.

As used herein, the term “PTPMEG modulator” includes both “activators” and “inhibitors” of PTPMEG phosphatase activity. An “activator” of PTPMEG is a substance that causes PTPMEG to become more active, and thereby directly or indirectly decreases the phosphotyrosine level and decreases activation of NMDA-R. The mode of action of the activator may be through binding PTPMEG; through binding another molecule which otherwise interacts with PTPMEG; etc. Conversely, an “inhibitor” of PTPMEG is a substance that causes PTPMEG to become less active, and thereby directly or indirectly increases activation of NMDA-R. The inhibition of PTPMEG may be complete or partial, and due to a direct or an indirect effect.

As used herein, the term “polypeptide containing the PDZ domain of PTPMEG” includes PTPMEG, and other polypeptides that contain the PDZ domain of PTPMEG, or their derivatives, analogs, variants, or fusion proteins that can bind to NR2A and/or NR2B. The term “polypeptide containing PTPMEG-binding site of NMDA-R” include an NMDA-R that has at least an NR2A or NR2B subunit, NR2A, NR2B, and other polypeptides that contain the PTPMEG-binding site of NR2A or NR2B, or their derivatives, analogs, variants, or fusion proteins that can bind to PTPMEG. Examples of PDZ domains are reviewed in Sheng and Sala (2001) Annu. Rev. Neurosci. 24:1-29, and Ponting et al. (1997) Bioessays 19:469-479.

As used herein, the term “PTPMEG/NMDA-R-containing protein complex” refers to protein complexes, formed in vitro or in vivo, that contain PTPMEG and NMDA-R. When only the binding of PTPMEG and NMDA-R is of concern, a polypeptide containing the PDZ domain of PTPMEG and a polypeptide containing PTPMEG-binding site of NMDA-R can substitute for PTPMEG and NMDA-R respectively. However, when dephosphorylation of NMDA-R is in concern, only a PTPMEG functional derivative and an NMDA-R functional derivative as defined herein can respectively substitute for PTPMEG and NMDA-R in the complex. The complex may also comprise other components, e.g., a protein tyrosine kinase, particularly Src.

The terms “substantially pure” or “isolated,” when referring to proteins and polypeptides, e.g., a fragment of PTPMEG, denote those polypeptides that are separated from proteins or other contaminants with which they are naturally associated. A protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least 75%, more preferably, at least 90%, of the total protein. Preferably, the protein will make up greater than about 90%, and more preferably, greater than about 95% of the total protein in the composition.

A “variant” of a molecule such as PTPMEG or NMDA-R is meant to refer to a molecule substantially similar in structure and biological activity to either the entire molecule, or to a fragment thereof. Thus, provided that two molecules possess a similar activity, they are considered variants as that term is used herein if the composition or secondary, tertiary, or quaternary structure of one of the molecules is not identical to that found in the other, or if the sequence of amino acid residues is not identical.

As used herein, “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide.

The term “operably linked” refers to functional linkage between a nucleic acid expression control sequence (such as a promoter, signal sequence, or array of transcription factor binding sites) and a second polynucleotide, wherein the expression control sequence affects transcription and/or translation of the second polynucleotide.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “heterologous sequence” or a “heterologous nucleic acid,” as used herein, is one that originates from a source foreign to the particular host cell, or, if from the same source, is modified from its original form. Thus, a heterologous gene in a prokaryotic host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding PTPMEG) and a promoter. Additional factors necessary or helpful in effecting expression can also be used as described herein. For example, transcription termination signals, enhancers, and other nucleic acid sequences that influence gene expression, can also be included in an expression cassette.

As used herein, “contacting” has its normal meaning and refers to combining two or more agents (e.g., two proteins, a polynucleotide and a cell, etc.). Contacting can occur in vitro (e.g., two or more agents [e.g., a test compound and a cell lysate] are combined in a test tube or other container) or in situ (e.g., two polypeptides can be contacted in a cell by coexpression in the cell, of recombinant polynucleotides encoding the two polypeptides), in a cell lysate”

Various biochemical and molecular biology methods referred to herein are well known in the art, and are described in, for example, Sambrook et al., Molecular Cloning: A Laboratory Manual, Cold Spring Harbor Press, N.Y. Second (1989) and Third (2000) Editions, and Current Protocols in Molecular Biology, (Ausubel, F. M. et al., eds.) John Wiley & Sons, Inc., New York (1987-1999).

### SCREENING FOR MODULATORS OF NMDA-R SIGNALING

The present invention provides methods for identifying modulators of NMDA-R signaling. The NMDA-R modulators are identified by detecting the ability of an agent to affect the activity PTPMEG. Surprisingly, it has been found that PTPMEG is capable of indirectly acting on NMDA-R. Active PTPMEG dephosphorylates Src at a residue critical for activity, thereby inactivating this protein kinase. Inactive Src cannot phosphorylate and activate the NMDA-R. PTPMEG also binds directly to NMDA-R and dephosphorylates it. As a result of these activities, the activity of NMDA-R is downregulated in the presence of active PTPMEG, and upregulated when PTPMEG is inhibited. In one embodiment, the NMDA-R modulators are screened for their ability to modulate PTPMEG phosphatase activity. In another embodiment, the NMDA-R modulators are identified by detecting their ability to promote or suppress the binding to PTPMEG and to NMDA-R.

As will be detailed below, PTPMEG, the NMDA-R/PTPMEG-containing protein complex, or cell lines and primary cultures that express PTPMEG or NMDA-R/PTPMEG-containing protein complex, are used to screen for PTPMEG agonists and antagonists that modulate direct or indirect NMDA-R tyrosine dephosphorylation, e.g. in the presence of Src protein tyrosine kinase. An agent that enhances the ability of PTPMEG to directly or indirectly dephosphorylate NMDA-R will result in a net decrease in the amount of phosphotyrosine on NMDA-R, whereas an agent that inhibits the ability of PTPMEG to directly or indirectly dephosphorylate NMDA-R will result in a net increase in the amount of phosphotyrosine on NMDA-R.

In some embodiments, the ability of an agent to enhance or inhibit NMDA-R activity is assayed in an in vitro system. In general, the in vitro assay format involves adding an agent to PTPMEG (or a functional derivative of PTPMEG) and NMDA-R, and measuring the biological activity or tyrosine phosphorylation level of the substrate (NMDA-R). Optionally, a protein tyrosine kinase, e.g. Fyn, Src, etc., usually Src, will also be present.

In other embodiments, the ability of an agent to enhance or inhibit Src activity is assayed in an in vitro system. In general, such as assay format involves adding an agent to PTPMEG (or a functional derivative of PTPMEG) and Src, and measuring the biological activity or tyrosine phosphorylation level of the substrate (Src protein). Optionally, a substrate of Src will also be present, e.g. NMDA-R.

In one embodiment of such assays, as a control, tyrosine phosphorylation level of the substrate is also measured under the same conditions except that the test agent is not present. By comparing the tyrosine phosphorylation levels of the substrate, PTPMEG antagonists or agonists can be identified. Specifically, a PTPMEG antagonist is identified if the presence of the test agent results in an increased tyrosine phosphorylation level of the substrate. Conversely, a decreased tyrosine phosphorylation level in the substrate indicates that the test agent is a PTPMEG agonist. The invention provides the use of such agents to modulate NMDA-R activity.

PTPMEG used in the assays is obtained from various sources. In some embodiments, PTPMEG used in the assays is purified from cellular or tissue sources, e.g., by immunoprecipitation with specific antibodies. In other embodiments, as described below, PTPMEG is purified by affinity chromatography utilizing specific interactions of PTPMEG with known protein motifs, e.g., the interaction of the PDZ domain of PTPMEG with NR2A and/or NR2B. In still other embodiments, PTPMEG, either holoenzyme or enzymatically active parts of it, is produced recombinantly either in bacteria or in eukaryotic expression systems. The recombinantly produced variants of PTPMEG can contain short protein tags, such as immunotags (HA-tag, c-myc tag, FLAG-tag) , 6×His-tag, GST tag, etc., which could be used to facilitate the purification of recombinantly produced PTPMEG using immunoaffinity or metal-chelation-chromatography, respectively. Polyclonal antibodies against PTPMEG using amino-terminal peptide: MTSRFRLPAGRTC and carboxy-terminal peptide CEGFVKPLTTSTNK have been generated.

Various substrates are used in the assays. Preferably, the substrate is Src, Fyn, NMDA-R, a functional derivative of NMDA-R, or the NR2A or NR2B subunit. In some embodiments, the substrates used are proteins purified from a tissue (such as immunoprecipitated NR2A or NR2B from rat brain). In other embodiments, the substrates are recombinantly expressed proteins. Examples of recombinant substrates include, but are not limited to, proteins expressed in E. coli, yeast, or mammalian expression systems. In still other embodiments, the substrates used are synthetic peptides that are tyrosine phosphorylated by specific kinase activity, e.g., Src or Fyn kinases.

Methods and conditions for expression of recombinant proteins are well known in the art. See, e.g., Sambrook, supra, and Ausubel, supra. Typically, polynucleotides encoding the phosphatase and/or substrate used in the invention are expressed using expression vectors. Expression vectors typically include transcriptional and/or translational control signals (e.g., the promoter, ribosome-binding site, and ATG initiation codon). In addition, the efficiency of expression can be enhanced by the inclusion of enhancers appropriate to the cell system in use. For example, the SV40 enhancer or CMV enhancer can be used to increase expression in mammalian host cells. Typically, DNA encoding a polypeptide of the invention is inserted into DNA constructs capable of introduction into and expression in an in vitro host cell, such as a bacterial (e.g., E. coli, Bacillus subtilus), yeast (e.g., Saccharomyces), insect (e.g., Spodoptera frugiperda), or mammalian cell culture systems. Mammalian cell systems are preferred for many applications. Examples of mammalian cell culture systems useful for expression and production of the polypeptides of the present invention include human embryonic kidney line (293; Graham et al., 1977, J. Gen. Virol. 36:59); CHO (ATCC CCL 61 and CRL 9618); human cervical carcinoma cells (HeLa, ATCC CCL 2); and others known in the art. The use of mammalian tissue cell culture to express polypeptides is discussed generally in Winnacker, FROM GENES TO CLONES (VCH Publishers, N.Y., N.Y., 1987) and Ausubel, supra. In some embodiments, promoters from mammalian genes or from mammalian viruses are used, e.g., for expression in mammalian cell lines. Suitable promoters can be constitutive, cell type-specific, stage-specific, and/or modulatable or regulatable (e.g., by hormones such as glucocorticoids). Useful promoters include, but are not limited to, the metallothionein promoter, the constitutive adenovirus major late promoter, the dexamethasone-inducible MMTV promoter, the SV40 promoter, and promoter-enhancer combinations known in the art.

The substrate may or may not be already in a tyrosine phosphorylated state (Lau & Huganir, J. Biol. Chem., 270: 20036-20041, 1995). In the case of a nonphosphorylated starting material, the substrate is typically phosphorylated, e.g., using an exogenous tyrosine kinase activity such as Src or Fyn.

A variety of standard procedures well known to those of skill in the art are used to measure the tyrosine phosphorylation levels of the substrates. In some embodiments, a phosphotyrosine-recognizing antibody-based assay is used, e.g., radioimmunoassay (RIA), enzyme-linked immunosorbent assay (ELISA), as well as fluorescently labeled antibodies whose binding can be assessed from levels of emitted fluorescence. See, e.g., U.S. Pat. No. 5,883,110; Mendoza et al., Biotechniques. 27: 778-788, 1999. In other embodiments, instead of immunoassays, the substrates are directly labeled with a radioactive phosphate group using kinases that carry out selective tyrosine phosphorylation (Braunwaler et al., Anal. Biochem. 234:23-26, 1996). The rate of removal of radioactive label from the labeled substrate can be quantitated in liquid (e.g., by chromatographic separation) or in solid phase (in gel or in Western blots).

Comparing a tyrosine phosphorylation level under two different conditions (e.g., in the presence and absence of a test agent) sometimes includes the step of recording the level of phosphorylation in a first sample or condition and comparing the recorded level with that of (or recorded for) a second portion or condition.

In some embodiments of the invention, the in vitro assays are performed with an NMDA-R/PTPMEG-containing protein complex. Such protein complexes contain NMDA-R and PTPMEG, or their functional derivatives. In addition, the complexes may also contain kinases, e.g. Fyn or Src, and other molecules. The NMDA-R/PTPMEG-containing protein complexes may be obtained from neuronal cells using methods well known in the art, e.g., immunoprecipitation as described in Grant et al. (WO 97/46877). Tyrosine phosphorylation levels of the substrates are assayed with standard SDS-PAGE and immunoblot analysis.

In other embodiments, NMDA-R signaling modulators of the present invention are also identified using in vivo assays. Such in vivo assay formats usually entail culturing cells co-expressing PTPMEG and its substrate (e.g., NR2A or NR2B; e.g., recombinant forms of PTPMEG and/or NMDA-R subunit substrate(s)), adding an agent to the cell culture, and measuring tyrosine phosphorylation level of the substrate in the cells. In one embodiment, as a control, tyrosine phosphorylation level of the substrate in cells not exposed to the test agent is also measured or determined.

In one embodiment, the in vivo screening system is modified from the method described in U.S. Pat. No. 5,958,719. Using this screening system, intact cells that express PTPMEG and one or more substrate(s) of PTPMEG (e.g., Src, Fyn, NMDA-R, NR2A, or NR2B) are first treated (e.g., by NMDA) to stimulate the substrate phosphorylation. The cells are then incubated with a substance that can penetrate into the intact cells and selectively inhibit further phosphorylation (e.g., by a PTK) of the substrate, e.g. NMDA-R. The degree of phosphorylation of the substrate is then determined by, for example, disrupting the cells and measuring phosphotyrosine level of the substrate according to methods described above, e.g. with standard SDS-PAGE and immunoblot analysis. The activity of PTPMEG is determined from the measured degree of phosphorylation of the substrate. An additional measurement is carried out in the presence of an agent. By comparing the degrees of phosphorylation, agonists or antagonist of PTPMEG that modulate NMDA-R tyrosine phosphorylation are identified.

In another embodiment, the present invention provides a method for identifying a nucleic acid molecule encoding a gene product that is capable of modulating the tyrosine phosphorylation level of NMDA-R. In one embodiment, a test nucleic acid is introduced into host cells coexpressing PTPMEG and NMDA-R or their functional derivatives. Methods for introducing a recombinant or exogenous nucleic acid into a cell are well known and include, without limitation, transfection, electroporation, injection of naked nucleic acid, viral infection, liposome-mediated transport (see, e.g., Dzau et al., 1993, Trends in Biotechnology 11:205-210; Sambrook, supra, Ausubel, supra). The cells are cultured so that the gene product encoded by the nucleic acid molecule is expressed in the host cells and interacts with PTPMEG and NMDA-R or their functional derivatives, followed by measuring the phosphotyrosine level of the NMDA-R. The effect of the nucleic acid on NMDA-R-signaling is determined by comparing NMDA-R phosphotyrosine levels measured in the absence or presence of the nucleic acid molecule.

It will be appreciated by one of skill in the art that modulation of binding of PTPMEG and NMDA-R may also affect the level of tyrosine phosphorylation in NMDA-R by PTPMEG. Therefore, agents identified from screening using the in vivo and in vitro assay systems described above may also encompass agents that modulate NMDA-R tyrosine phosphorylation by modulating the binding of PTPMEG and NMDA-R. In some embodiments of the invention, NMDA-R modulators are identified by directly screening for agents that promote or suppress the binding of PTPMEG and NMDA-R. Agents thus identified may be further examined for their ability to modulate NMDA-R tyrosine phosphorylation, using methods described above or standard assays well known in the art.

A variety of binding assays are useful for identifying agents that modify the interaction between the PDZ domain of PTPMEG and NR2A (or NR2B). In certain embodiments, two-hybrid based assays are used.

The cDNAs encoding the C-terminal portion, typically at least 100, 200, 400, or 600 C-terminal amino acid residues, of NR2A or NR2B and at least the PDZ domain of PTPMEG are cloned into yeast two-hybrid vectors encoding the DNA binding domain and DNA activation domain, respectively, or vice-versa. The yeast two-hybrid used is based on the yeast GAL4 transcriptional system (Song & Fields, Nature 340: 245-246, 1989), the Sos-Ras complementation system (Aronheim et al., Mol. Cell. Biol. 17: 3094-3102, 1997), the bacterial LexA transcriptional system (Current Protocols in Mol. Biol., Ausubel et al. Eds, 1996, New York), or any other system of at least equal performance. Reporter gene constructs, such as α- or β-galactosidase, β-lactamase, or green fluorescent protein (see Tombolini et al., Methods Mol Biol. 102: 285-98, 1998; Kain et al., Methods Mol Biol. 63: 305-24, 1997), are produced using necessary regulatory elements from promoter regions of above-mentioned transcription factors. Alternatively, modular signaling molecules are engineered to be brought together by the interaction between NR2A and/or NR2B and PTPMEG in the Sos-Ras complementation-based yeast two-hybrid system. These constructs are transiently or stably transformed into a yeast strain to be used in the screen.

In one embodiment, the GAL4 system is used to screen agents that modulate the binding of PTPMEG and NMDA-R. DNA binding domain vector containing the C-terminal portion of NR2A or NR2B and DNA activation domain vector containing the PDZ domain of PTPMEG are cotransformed into the same yeast strain which carries one of the reporters. The interaction between PTPMEG and NMDA-R activates the expression of the reporter gene. The yeast culture in which the reporter genes is expressed is divided in equal amounts to 96- or 384-well assay plates. The levels of α- or β-galactosidase, β-lactamase are measured by quantifying their enzymatic activity using colorimetric substrates, such as orthomethylphenylthiogalactoside (OMTP) or X-gal; the levels of GFP are assessed fluorometrically. Pools of agents or individual agents are added to yeast cultures in wells and the levels of inhibition or facilitation of the interaction by the agents are determined from the levels of the reporter gene activity. Agents that decrease the reporter gene expression are antagonists of the interaction between PTPMEG and NR2A or NR2B. In contrast, agents that facilitate the reporter gene expression are agonists of the interaction between PTPMEG and NR2A or NR2B.

The bacterial two-hybrid screening system is based on the reconstitution, in an Escherichia coli cya strain, of a signal transduction pathway that takes advantage of the positive control exerted by cAMP (Karimova et al., Proc. Natl. Acad. Sci. USA. 95:5752-56, 1998). Association of the two-hybrid proteins, such as that of PTPMEG with NR2A and/or NR2B, results in functional complementation between T25 and T18 fragments and leads to cAMP synthesis. Cyclic AMP then triggers transcriptional activation of catabolic operons, such as lactose or maltose, which yield a characteristic phenotype.

The mammalian two-hybrid assay is also based on transcriptional activation. See, The Yeast Two-Hybrid System. Bartel & Fields, Eds. 1997, Oxford, Oxford University Press. In the present invention, the cDNAs encoding at least the C-terminal portion of NR2A or NR2B and at least the PDZ domain of PTPMEG are cloned into mammalian two-hybrid vectors encoding the DNA binding domain and the VP16 DNA activation domain, respectively, or vice-versa. These vector constructs are co-transfected into the cell line which harbors a reporter gene (CAT, luciferase, GFP, α- or β-galactosidase, β-lactamase) under the control of the VP16 responsive promoter. Transcriptional activation in cells reflected by the levels of the reporter gene or its activity is proportional to the strength of interaction between the C-terminal portions of NR2A or NR2B and the PDZ domain of PTPMEG. The cell culture in which the reporter gene is expressed is divided in equal amounts to 96- or 384-well assay plates. The expression levels of CAT, α- or β-galactosidase, β-lactamase are measured by quantifying their enzymatic activity using colorimetric substrates, such as X-gal; the levels of GFP or luciferase are assessed fluorometrically or spectrophotometrically, respectively. Agents that modulate the PTPMEG binding to NR2A and/or NR2B are similarly identified as that described in the yeast two-hybrid assay.

In some embodiments of the invention, agents (e.g., peptides) that bind to the PDZ domain of PTPMEG with high affinity are identified by phage display, an oriented peptide library approach (Songyang et al., Science 275: 73-77, 1997) or a lacI repressor system (Stricker et al., Methods in Enzymology 303: 451-468, 1999). These peptides are further screened for their ability to modulate the interaction between PTPMEG and NR2A or NR2B.

In one embodiment, modulators of the interaction between PTPMEG and NR2A or NR2B are identified by detecting their abilities to either inhibit PTPMEG and NMDA-R from binding (physically contacting) each other or disrupts a binding of PTPMEG and NMDA-R that has already been formed. The inhibition or disruption can be either complete or partial. In another embodiment, the modulators are screened for their activities to either promote PTPMEG and NMDA-R binding to each other, or enhance the stability of a binding interaction between PTPMEG and NMDA-R that has already been formed. In either case, some of the in vitro and in vivo assay systems discussed above for identifying agents which modulate the NMDA-R tyrosine phosphorylation level may be directly applied or readily modified to monitor the effect of an agent on the binding of NMDA-R and PTPMEG. For example, a cell transfected to coexpress PTPMEG and NMDA-R or receptor subunit, in which the two proteins interact to form an NMDA-R/PTPMEG-containing complex, is incubated with an agent suspected of being able to inhibit this interaction, and the effect on the interaction measured. In some embodiments, a polypeptide containing the PDZ domain of PTPMEG and a polypeptide containing PTPMEG-binding site of NMDA-R can substitute for the intact PTPMEG and NMDA-R proteins, respectively, in the NMDA-R/PTPMEG-containing protein complexes. Any of a number of means, such as coimmunoprecipitation, is used to measure the interaction and its disruption.

Although the foregoing assays or methods are described with reference to PTPMEG and NMDA-R, the ordinarily skilled artisan will appreciate that functional derivatives or subunits of PTPMEG and NMDA-R may also be used. For example, in various embodiments, NR2A or NR2B is used to substitute for an intact NMDA-R in assays for screening agents that modulate binding of PTPMEG and NMDA-R. In a related embodiment, an NMDA-R, Src, Fyn, functional derivative is used for screening agents that modulate PTPMEG phosphatase activity. In another embodiment, a polypeptide containing the PDZ domain of PTPMEG is used for screening agents that modulate the binding of PTPMEG and NMDA-R.

Further, in various embodiments, functional derivatives of PTPMEG that have amino acid deletions and/or insertions and/or substitutions (e.g., conservative substitutions) while maintaining their catalytic activity and/or binding capacity are used for the screening of agents. A functional derivative is prepared from a naturally occurring or recombinantly expressed PTPMEG and NMDA-R by proteolytic cleavage followed by conventional purification procedures known to those skilled in the art. Alternatively, the functional derivative is produced by recombinant DNA technology by expressing only fragments of PTPMEG or NMDA-R in suitable cells. In one embodiment, the partial receptor or phosphatase polypeptides are expressed as fusion polypeptides. It is well within the skill of the ordinary practitioner to prepare mutants of naturally occurring NMDA/PTPMEG proteins that retain the desired properties, and to screen the mutants for binding and/or enzymatic activity. Typically, functional derivatives of NMDA-R subunits NR2A and NR2B that bind PTPMEG will include the “tSXV motif” of these subunits. NR2A and NR2B derivatives that can be dephosphorylated typically comprise the cytoplasmic domain of the polypeptides, e.g., the C-terminal 900 amino acids or a fragment thereof. Deletion constructs and binding domains are published in Hironaka et al. Functional derivatives that retain enzymatic (dephosphorylation) activity include the C-terminal PTP domain.

In some embodiments, cells expressing PTPMEG and NMDA-R may be used as a source of PTPMEG and/or NMDA-R, crude or purified, or in a membrane preparation, for testing in these assays. Alternatively, whole live or fixed cells may be used directly in those assays. Methods for preparing fixed cells or membrane preparations are well known in the art, see, e.g., U.S. Pat. No. 4,996,194. The cells may be genetically engineered to coexpress PTPMEG and NMDA-R. The cells may also be used as host cells for the expression of other recombinant molecules with the purpose of bringing these molecules into contact with PTPMEG and/or NMDA-R within the cell.

### THERAPEUTIC APPLICATIONS AND PHARMACEUTICAL COMPOSITIONS

It is well known in the art that NMDA-R agonists and antagonists can be used to treat symptoms caused by abnormal NMDA-R signaling, e.g. acute insult of the central nervous system (CNS). Methods of treatment using pharmaceutical composition comprising NMDA agonists and/or NMDA antagonists have been described, e.g., in U.S. Pat. No. 5,902,815.

As discussed in detail below, the present invention provides pharmaceutical compositions containing PTPMEG antagonists and/or agonists that modulate NMDA-R tyrosine phosphorylation. Such agonists and antagonists include, but are not limited to, agents that interfere with PTPMEG gene expression, agents that modulate the ability of PTPMEG to bind to NMDA-R or to dephosphorylate NMDA-R. In one embodiment, a PTPMEG antisense oligonucleotide is used as a PTPMEG antagonist in the pharmaceutical compositions of the present invention. In addition, PTP inhibitors that inhibit PTPMEG dephosphorylation of NMDA-R are useful as NMDA-R signaling modulators (e.g., dephostatin, orthovanadate, Li et al., Biochim. Biophys. Acta. 1405:110-20, 1998).

Abnormal NMDA-R activity elicited by endogenous glutamate is implicated in a number of important CNS disorders. In one aspect, the present invention provides activators of PTPMEG that, by decreasing phosphotyrosine level of NMDA-R, can treat or alleviate symptoms mediated by abnormal NMDA-R signaling. Indications of interest include mild cognitive impairment (MCI), which can progress to Alzheimer's disease (AD). Treatment with acetylcholinesterase inhibitors can provide for modest memory improvement. Cognitive enhancers may also find use for memory loss associated with aging, and in the general public. One important use for NMDA activator drugs involves the ability to prevent or reduce excitotoxic damage to neurons. In some embodiments, the PTPMEG activators of the present invention, which promote the dephosphorylation of NMDA-R, are used to alleviate the toxic effects of excessive NMDA-R signaling.

In certain other embodiments, PTPMEG inhibitors of the present invention, which function as NMDA-R agonists, are used therapeutically to treat conditions caused by NMDA-R hypo-function, i.e., abnormally low levels of NMDA-R signaling in CNS neurons. NMDA-R hypofunction can occur as an endogenous disease process. It can also occur as a drug-induced phenomenon, following administration of an NMDA antagonist drug. In some related embodiments, the present invention provides pharmaceutical compositions containing PTPMEG inhibitors that are used in conjunction with NMDA agonists, e.g., to prevent the toxic side effects of the NMDA antagonists.

Excessive glutamatergic signaling has been causatively linked to the excitotoxic cell death during an acute insult to the central nervous system such as ischemic stroke (Choi et al., Annu Rev Neurosci. 13: 171-182, 1990; Muir & Lees, Stroke 26: 503-513, 1995). Excessive glutamatergic signaling via NMDA receptors has been implicated in the profound consequences and impaired recovery after the head trauma or brain injury (Tecoma et al., Neuron 2:1541-1545, 1989; Mcintosh et al., J. Neurochem. 55:1170-1179, 1990). NMDA receptor-mediated glutamatergic hyperactivity has also been linked to the process of slow degeneration of neurons in Parkinson's disease (Loopuijt & Schmidt, Amino Acids, 14: 17-23, 1998) and Huntington's disease (Chen et al., J. Neurochem. 72:1890-1898, 1999). Further, elevated NMDA-R signaling in different forms of epilepsy have been reported (Reid & Stewart, Seizure 6: 351-359, 1997).

Accordingly, PTPMEG activators of the present invention are used for the treatment of these diseases or disorders by stimulating the NMDA receptor-associated phosphatase activity (such as that of PTPMEG) or by promoting the binding of PTPMEG to the NMDA receptor complex.

The PTPMEG agonists (inhibitors of NMDAR activity) of the present invention can also be used to treat diseases where a mechanism of slow excitotoxicity has been implicated (Bittigau & Ikonomidou, J. Child. Neurol. 12: 471-485, 1997). These diseases include, but are not limited to, spinocerebellar degeneration (e.g., spinocerebellar ataxia), motor neuron diseases (e.g., amyotrophic lateral sclerosis (ALS)), mitochondrial encephalomyopathies. The PTPMEG agonists of the present invention can also be used to alleviate neuropathic pain, or to treat chronic pain without causing tolerance or addiction (see, e.g., Davar et al., Brain Res. 553: 327-330, 1991).

NMDA-R hypofunction has also been causatively linked to schizophrenic symptoms (Tamminga, Crit. Rev. Neurobiol. 12: 21-36, 1998; Carlsson et al., Br. J. Psychiatry Suppl.: 2-6, 1999; Corbett et al., Psychopharmacology (Berl). 120: 67-74, 1995; Mohn et al., Cell 98: 427-436, 1999) and various forms of cognitive deficiency, such as dementias (e.g., senile and HIV-dementia) and Alzheimer's disease (Lipton, Annu. Rev. Pharmacol. Toxicol. 38:159-177, 1998; Ingram et al., Ann. N.Y. Acad. Sci. 786: 348-361, 1996; Müller et al., Pharmacopsychiatry. 28: 113-124, 1995). In addition, NMDA-R hypofunction is also linked to psychosis and drug addiction (Javitt & Zukin, Am J Psychiatry. 148: 1301-8, 1991). Further, NMDA-R hypofunction is also associated with ethanol sensitivity (Wirkner et al., Neurochem. Int. 35: 153-162, 1999; Yagi, Biochem. Pharmacol. 57: 845-850, 1999).

NMDA receptor hypofunction has long been implicated in the etiology of depression. For a review, see Schiffer (2002) Mol Neurobiol. 25(2):191-212. Many antidepressant drugs show activity at the NMDA receptor.

Using PTPMEG antagonists (activators of NMDAR) described herein, the present invention provides methods for the treatment of schizophrenia, psychosis, depression, cognitive deficiencies, drug addiction, and ethanol sensitivity by antagonizing the activity of the NMDA-R-associated PTPs, and that of PTPMEG in particular, or by inhibiting the interaction between PTPMEG and the NR2A or NR2B subunit.

The PTPMEG agonists and antagonists of the present invention are directly administered under sterile conditions to the host to be treated. However, while it is possible for the active ingredient to be administered alone, it is often preferable to present it as a pharmaceutical formulation. Formulations typically comprise at least one active ingredient together with one or more acceptable carriers thereof. Each carrier should be both pharmaceutically and physiologically acceptable in the sense of being compatible with the other ingredients and not injurious to the patient. For example, the bioactive agent may be complexed with carrier proteins such as ovalbumin or serum albumin prior to their administration in order to enhance stability or pharmacological properties such as half-life. Furthermore, therapeutic formulations of this invention are combined with or used in association with other therapeutic agents.

The therapeutic formulations are delivered by any effective means that could be used for treatment. Depending on the specific NMDA-R antagonist and/or NMDA-R agonist being used, the suitable means include but are not limited to oral, rectal, nasal, pulmonary administration, or parenteral (including subcutaneous, intramuscular, intravenous and intradermal) infusion into the bloodstream.

Therapeutic formulations are prepared by any methods well known in the art of pharmacy. See, e.g., Gilman et al (eds.) (1990) Goodman and Gilman's: The Pharmacological Bases of Therapeutics (8th ed.) Pergamon Press; and (1990) Remington's Pharmaceutical Sciences (17th ed.) Mack Publishing Co., Easton, Pa.; Avis et al (eds.) (1993) Pharmaceutical Dosage Forms: Parenteral Medications Dekker, N.Y.; Lieberman et al. (eds.) (1990) Pharmaceutical Dosage Forms: Tablets Dekker, N.Y.; and Lieberman et al (eds.) (1990) Pharmaceutical Dosage Forms: Disperse Systems Dekker, N.Y. The therapeutic formulations can conveniently be presented in unit dosage form and administered in a suitable therapeutic dose. The preferred dosage and mode of administration of a PTPMEG agonist and/or antagonist will vary for different patients, depending upon factors that will need to be individually reviewed by the treating physician. As a general rule, the quantity of a PTPMEG agonist and/or antagonist administered is the smallest dosage which effectively and reliably prevents or minimizes the conditions of the patients.

A suitable therapeutic dose is determined by any of the well known methods such as clinical studies on mammalian species to determine maximum tolerable dose and on normal human subjects to determine safe dosage. In human patients, since direct examination of brain tissue is not feasible, the appearance of hallucinations or other psychotomimetic symptoms, such as severe disorientation or incoherence, should be regarded as signals indicating that potentially neurotoxic damage is being generated in the CNS by an NMDA-R antagonist. Additionally, various types of imaging techniques (such as positron emission tomography and magnetic resonance spectroscopy, which use labeled substrates to identify areas of maximal activity in the brain) may also be useful for determining preferred dosages of NMDA-R agonists for use as described herein, with or without NMDA-R antagonists.

It is also desirable to test rodents or primates for cellular manifestations in the brain, such as vacuole formation, mitochondrial damage, heat shock protein expression, or other pathomorphological changes in neurons of the cingulate and retrosplenial cerebral cortices. These cellular changes can also be correlated with abnormal behavior in lab animals.

Except under certain circumstances when higher dosages may be required, the preferred dosage of a PTPMEG agonist and/or antagonist will usually lie within the range of from about 0.001 to about 1000 mg, more usually from about 0.01 to about 500 mg per day. It should be understood that the amount of any such agent actually administered will be determined by a physician, in the light of the relevant circumstances that apply to an individual patient (including the condition or conditions to be treated, the choice of composition to be administered, including the particular PTPMEG agonist or the particular PTPMEG antagonist, the age, weight, and response of the individual patient, the severity of the patient's symptoms, and the chosen route of administration). Therefore, the above dosage ranges are intended to provide general guidance and support for the teachings herein, but are not intended to limit the scope of the invention.

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which scope will be determined by the language in the claims.

It must be noted that as used herein and in the appended claims, the singular forms “a”, “and”, and “the” include plural referents unless the context clearly dictates otherwise. Thus, for example, reference to “a mouse” includes a plurality of such mice and reference to “the cytokine” includes reference to one or more cytokines and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for all relevant purposes, e.g., the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

### Identification of Interaction Between NMDA-R and PTPMEG

Co-immunoprecipitation experiments demonstrating the NMDA-R/PTPMEG interaction were performed as follows. The combinations of eukaryotic CMV promoter driven expression vectors that contain cDNAs encoding the following proteins are co-expressed in 293 cells in different combinations.

Full Length Clones:

1. NR1,
2. NR2A,
3. NR2B
4. PTPMEG (amino-terminal myc-tag, c-terminal HA tag)
5. PTPMEG-CS (inactive PTPase) amino-terminal myc-tag, c-terminal HA tag)

For all experiments, 7-10 micrograms of total plasmid DNA per semi-confluent dish of cells were transfected by, e.g., calcium phosphate precipitation (Wigler M, et al., Cell 16:777-785, 1979). Cells can be harvested 48 hours post-transfection, the medium removed upon centrifugation and the cells resuspended in Lysis Buffer (150 mM NaCl, 50 mM Tris pH 7.6, 1% Triton). 200 μg lysate (1 μg/μl) is incubated with 1-3 μg of primary antibody, overnight at 4° C., shaking.

After co-incubation of antibodies and heterologously expressed proteins, 20 μl of Protein A/G Plus-Agarose (Santa Cruz) slurry was added, and the incubation was continued for another hour. To determine co-immunoprecipitated proteins, material bound to Protein AG-Plus Agarose was separated by pelleting the beads with the immunocomplex attached by centrifugation, washed with PBS and resolved by 4-12% SDS-PAGE. Proteins resolved on the gel were transferred to membrane to verify the presence of co-immunoprecipitated proteins by Western blots using specific antibodies as outlined above.

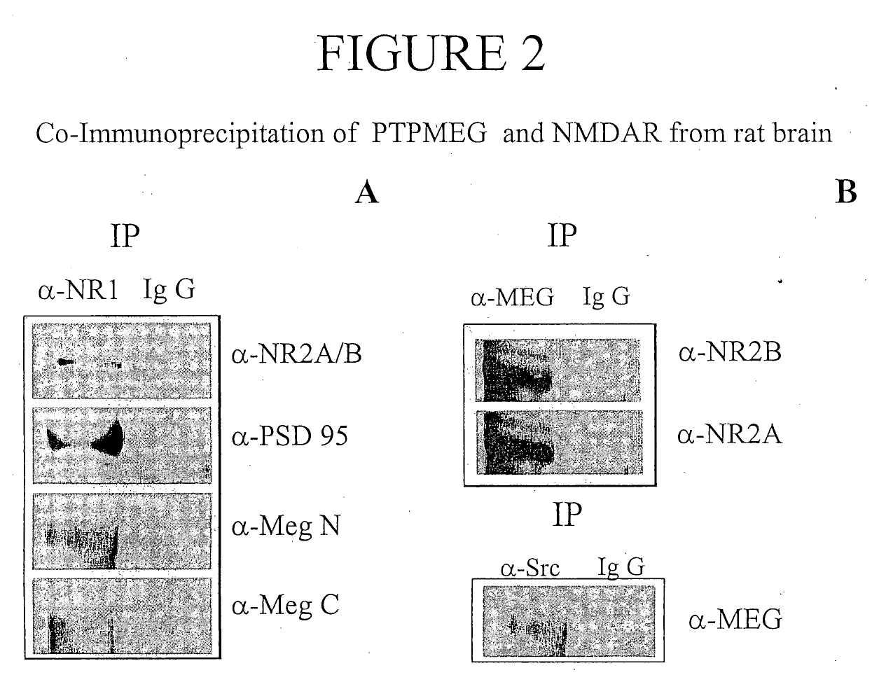

The data show that tagged full length PTPMEG co-precipitates with NR2 subunits and thus interacts with the receptor complex, as shown in FIG. 2.

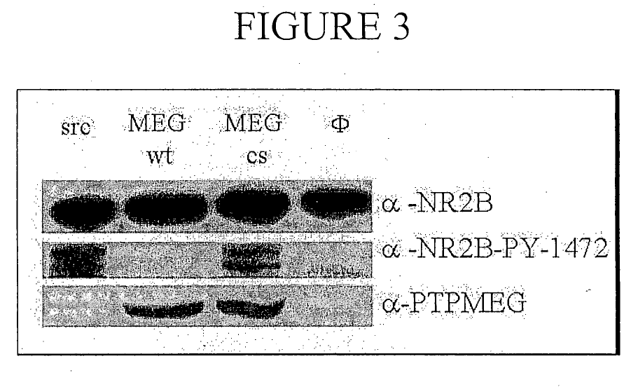

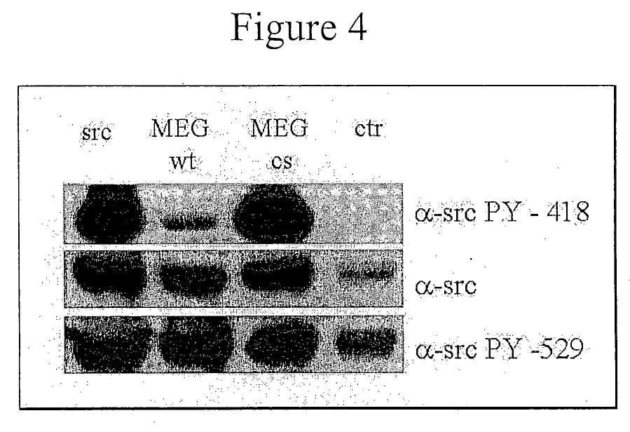

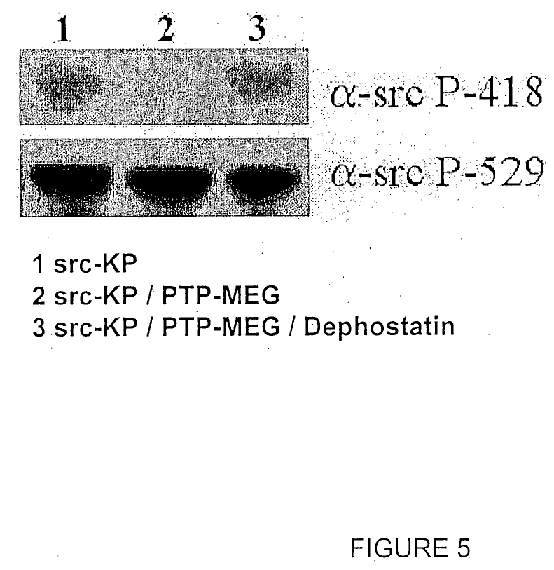

A stable cell line, expressing the NMDA receptor subunit NR1 together with NR2B was transiently transfected with constitutive active Src kinase and PTPMEG, wildtype and dominant negative CS mutant, respectively. Control experiments included dephostatin, a generic tyrosine phosphatase inhibitor. 48 h post transfection cells were lysed and subsequently analyzed by western blot. In presence of the active wildtype PTPMEG phosphatase, both the NR2B subunit as well as Src kinase were dephosphorylated, shown in FIG. 3. Inactivation of Src kinase by PTPMEG was shown using anti PY418 antibody to inactivate Src kinase at position 418 in a site specific manner, shown in FIG. 4. NR2B phosphorylation by Src kinase was detected by an NR2B phosphospecific antibody. Dephosphorylation of NR2B and Src kinase was reversed by addition of dephostatin, and unaffected in presence of the dominant negative form of PTPMEG (CS). (FIG. 5)

PTPMEG Dephosphorylates Protein Kinases Src and fyn. A stable cell line expressing the NR1 and NR2B subunits of the NMDA receptor was transiently transfected with constitutive active Src Srcor constitutive active fyn (fyn Y530F, where the tyrosine at position 530 is substituted by a phenylalanine) and PTPMEG, from a full length PTPMEG (amino-terminal myc-tag, c-terminal HA tag) expression construct in the pRK5 vector, with a CMV-driven promoter.

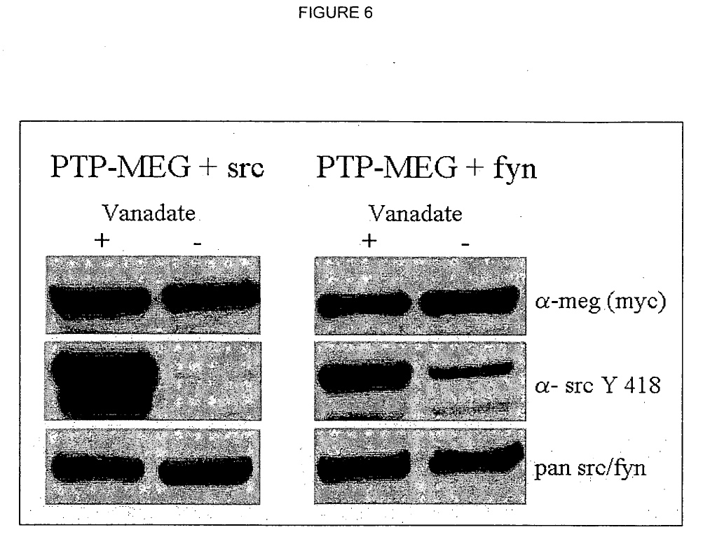

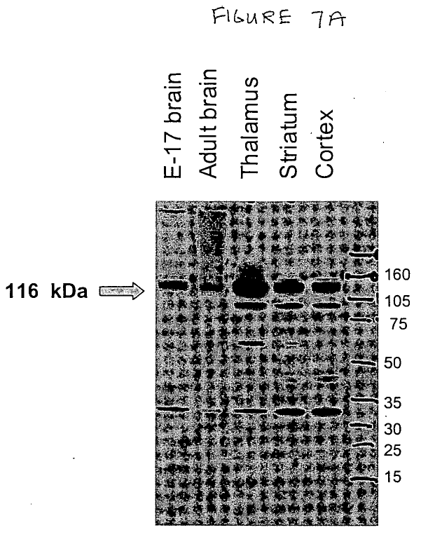

48 hrs post-transfection the cells were stimulated with 500 μM sodium orthovanadate, a generic tyrosine phosphatase for 3 hours. Cells were lysed and analyzed by western blot using a Src/fyn specific phosphoantibody (from Biosource) that recognizes the residue Y418 critical for activity for both Src and fyn. In presence of PTPMEG (identified with a c-myc antibody from Upstate) there is a dephosphorylation of Src and fyn at position Y418. This dephosphorylation is reversed by sodium orthovanadate. A pan Src antibody that recognizes total Src and fyn is used as a loading control. The results are shown in FIG. 6.

These data demonstrate that PTPMEG dephosphorylates Src and fyn at the tyrosine residue that is critical for activity (Y 418).

### Example 2

### Characterization of PTPMEG and NMDA-R

Expression

A 209 bp fragment was amplified from rat brain cDNA using (SEQ ID NO:1) PTPMEG 3120F (CCACTCTGAAGAAGGAAACACTGC) and (SEQ ID NO:2) PTPMEG 3329R (CCAGTTCTTCCGATTCCAGCAC). This fragment contains the 3′ end of the PTPMEG PTP domain. The RT-PCR fragment was cloned into PCR4TOPO, confirmed by sequencing and subsequently used to generate riboprobes for in-situ hybridization on rat brain tissue.

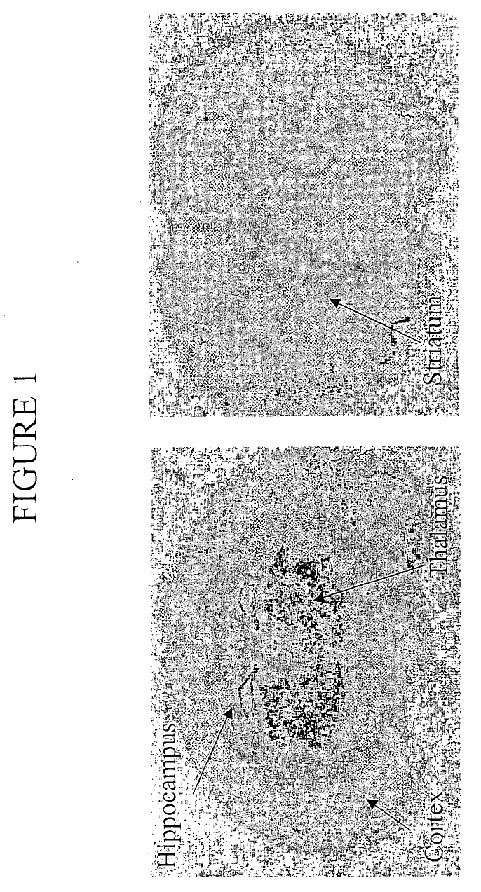

The results indicate that PTPMEG is expressed in all major neuronal populations in the adult rat brain (see FIG. 1). Thus, there is a very high degree of overlap between the cellular localization of PTPMEG and NMDA-R in the brain. In addition, PTPMEG expression was profiled in MCAO and global ischemia, as well as ischemic preconditioning. In global ischemia PTPMEG mRNA is upregulated after 24 h, compared to sham treated animals (sham+2 days recovery+10 min of ischemia). Upregulation is most prominent in the hippocampus as well as thalamic regions.

Animal Preparation and experimental Groups. The procedures for transient MCAO were performed as described previously (Zhao et al. (1997) J Cereb Blood Flow Metab. 17(12):1281-90) and are summarized briefly below. Male Wistar rats (Möllegaards Breeding Center, Copenhagen), weighing 310-350 g, were fasted overnight but had free access to water. Anesthesia was induced by inhalation of 3% halothane in N₂O:O₂ (70%:30%), whereafter the animals were intubated. They were then ventilated on 1.0-1.5% halothane in N₂O:O₂ during operation. The tail artery was cannulated for blood sampling and blood pressure monitoring. Blood pressure, PaO₂, PaCO₂, pH, and blood glucose were measured, and 0.1 ml of heparin (300 units×ml⁻¹) was given through the tail artery just before induction of ischemia. A surgical mid-line incision was made to expose the right common, internal, and external carotid arteries. The external carotid artery was ligated. The common carotid artery was closed by a ligature, and the internal carotid artery was temporarily closed by a microvascular clip. A small incision was made in the common carotid artery, and a nylon filament, which had a distal cylinder of silicon rubber (diameter 0.28 mm), was inserted into the internal carotid artery through the common carotid artery. The filament was further advanced 19 mm to occlude the origin of the middle cerebral artery (MCA). When the middle cerebral artery occlusion (MCAO) had been performed, animals were extubated and allowed to wake up and resume spontaneous breathing. In the group aimed for recirculation, the animals were reanesthetized with halothane after 2 hrs of MCAO, and the filament was withdrawn. During the operation, an electrical temperature probe was inserted 7 cm into the rectum to monitor core temperature, which was regularly maintained at 37° C. After the operation, the animals were cooled by an air cooling system to avoid the hypothermia which would otherwise occur and to keep core temperature close to normal levels during and following MCAO. All animals were tested for neurological status according to the neurological examination grading system described by Bederson et al. (1986) Stroke 17(3):472-6.

Animals were sacrificed after 2 h. of MCAO; or 3 min of ischemia for IPC and the time points as noted. The brain were taken out and frozen in imbedding media at −50° C. and stored at −80° C. before sectioning.

PTPMEG was examined by in situ hybridization. Tissue sections (15 μm) were cut on a Microm cryostat and thaw-mounted on positively charged slides. After fixation with 4% paraformaldehyde (4° C., 5 minutes), sections were processed as followed: 1) washed 2 minutes in 0.1 mol/L phosphate buffer saline (PBS pH 7.2. 2) 0.1 M TEA 1 minute. 3) 0.25% acetic anhydride\TEA for 10 minutes. 4) Rinse 2 times in SSC. 5) Dehydrated in 70% (two minutes), 95% (two minutes) and 100% (two minutes) ethanol. 6) 5 minutes in chloroform and 2 minutes in 95% ethanol and finally air-dried for 10 minutes. A solution containing labeled probes was then contacted with the cells and the probes allowed to hybridize. Excess probe was digested, washed away and the amount of hybridized probe measured.

The tissue from 2 h MCAO and 0, 1.5, 3, 6, 12, 24, and 48 hours recovery, and global ischemic preconditioning (IPC) (a model for tolerance to ischemic, see Shamloo and Wieloch (1999) J Cereb Blood Flow Metab 19(2):173-83) were generated and sectioned (3 min of ischemia (IPC) and 4 h, 12 h, 18 h, 24 h, and 48 h). Also sectioned were 10 min of ischemia with or without IPC (2 days before the 10 min) and 12 h, 18 h and 48 h of recovery (after the 10 min). The tissue sections were processed and stored at AGY tissue bank.

A PCR fragment was generated with SP6 and T7 promoter sequences for in vitro transcription (see Logel et al. (1992) Biotechniques 13(4):604-10. The amplified product was then used as a templicate for transcription to generate labeled mRNA, both sense and anti-sense. These probes were then used to hybridize to the tissue sections. Both sense and anti sense probes were generated and hybridized with MCAO or IPC tissues. Data were analyzed and information was stored.

These results show upregulation of PTPMEG mRNA in global ischemia, as well as IPC.

Immunocytochemistry. In primary neuronal culture derived from the rat cerebral cortex and hippocampus, studies of co-localization are conducted with the recombinantly expressed PTPMEG. A Sinbis virus carrying a full length myc and HA tagged PTPMEG cDNA is used to infect primary neurons. Clustering was observed in dendritic processes, which serve as input receivers from other cells and where NMDA-R are localized. The co-localization of PTPMEG and NMDA-R is demonstrated by immunocytochemistry using anti-NMDA-R antibodies.

High resolution immunohistochemistry studies on brain slices (50-200 micrometers in thickness) are carried out to demonstrate the subcellular co-localization as described in Antibodies, Harlow & Lane, Eds., 1999. Using NR1- and PTPMEG-specific antibodies to label endogenous NMDA-R and PTPMEG in neurons, the co-localization is detected by using antibodies derived from different species (such as rabbit or mouse; rabbit or goat etc). The secondary antibodies which carry different reporters (e.g., different fluorescent tags) and specifically recognize antibodies from a particular species are used to differentiate between NMDA-R and PTPMEG.

Antibody generation. Two polyclonal antibodies against PTPMEG using oligopeptides (SEQ ID NO:3) MTSRFRLPAGRTC and (SEQ ID NO:4) CEGFVKPLTTSTNK have been generated. Oligopeptide sequences were picked based on antigenicity prediction and an absence of potential glycosylation sites.

Modulation of NMDA-R signaling by PTPMEG. The following experiments are conducted to determine the role of PTPMEG in the modulation of NMDA-R signaling. Primary hippocampal neurons are transfected with or without PTPMEG and GFP as a marker using 5 micrograms of total plasmid DNA per well. The neurons co-expressing all components respond with the NMDA-R selective current when exposed to L-glutamate or NMDA. In order to measure NMDA currents, the cells are clamped with the patch pipette and characteristic NMDA-R currents recorded at different membrane potentials (Köhr & Seeburg, J. Physiol (London) 492: 445-452, 1996). Purified Src or Fyn is then allowed to diffuse to the cytosol of clamped cells through the patch pipette. Once again, the NMDA currents are recorded and the potentiation by the tyrosine kinases of NMDA-R currents is determined both in the presence and absence of transfected PTPMEG.

Alternatively, instead of applying purified Src or Fyn, a peptide, (SEQ ID NO:5) EPQ(pY)EEIPIA, that activates the members of Src family of tyrosine kinases is used to activate endogenous kinases in the cell and the NMDA-R currents are determined both in the presence and absence of transfected PTPMEG.

Patch clamp experiments with cells expressing NMDA-R and PTPMEG are carried out in the presence of 0.5 mM synthetic inhibitory peptides corresponding to the C-terminal nine amino acids of NR2A or NR2B (SEQ ID NO:6) (KLSSIESDV), as well as control peptides corresponding to the scrambled peptides with the same amino acid composition as the inhibitory peptide.

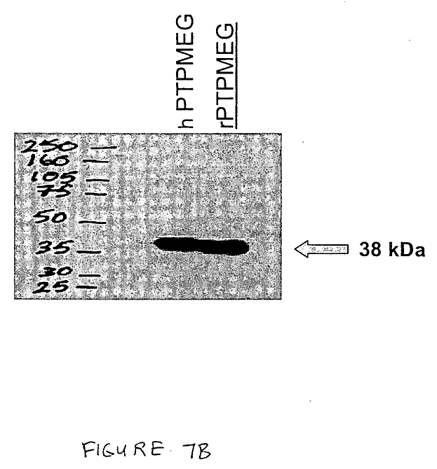

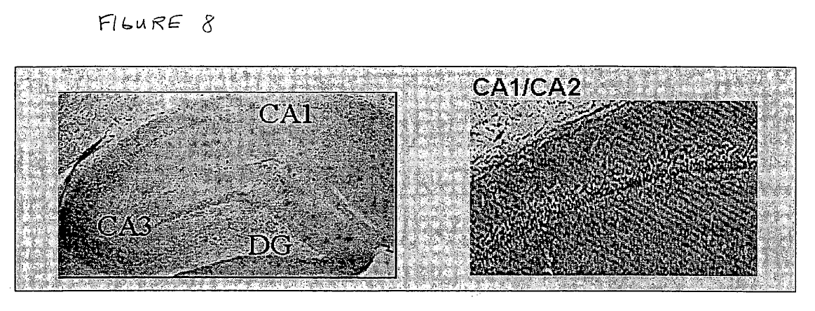

A Western blot was performed using lysates from primary neuronal cells derived from in different brain regions. The lysates were electrophoresed and blotted, and exposed to a C-terminal polyclonal antibody raised against the carboxy-terminal peptide: (SEQ ID NO:7) CEGFVKPLTTSTNK, which recognizes a 116 Kda protein that corresponds to PTPMEG. The antibody recognizes cleaved PTPMEG fragments in different brain regions (FIG. 7A) and both the human and rat PTPMEG catalytic domains (FIG. 7B). As shown in FIG. 7A, the lanes are as follows:
E-17 brain: lysate from a brain from a day 17 rat embryo
Adult brain: lysate from an adult rat brain
Thalamus, Striatum and Cortex: These are primary neuronal cultures derived from an E-17 rat embryo brain where the different regions (thalamus, cortex and striatum) have been dissected, digested and cultured for 14 days in vitro.

It has been shown (Gu M and Majerus P W. JBC 271, 27751-27759, 1996) that PTPMEG can be cleaved and activated by calpain in vivo and in vitro. Some of these fragments such as the 55 KDa have phosphatase activity.

Immunohistochemistry experiments using the PTPMEG C-terminal antibody show that the PTPMEG protein is found in the CA1/CA2 regions of the hippocampus. High resolution immunohistochemistry studies on brain slices (50-200 micrometers in thickness) are carried out to demonstrate the subcellular co-localization as described in Antibodies, Harlow & Lane, Eds., 1999, using PTPMEG-specific antibodies to label endogenous PTPMEG in hippocampus slices. PTPMEG is also present in the CA3 and DG regions.

De-Phosphorylation of NR2A or NR2B by PTPMEG. The following experiments are conducted to determine the role of PTPMEG in the modulation of NMDA-R signaling. Stable HEK293 cell lines (NR1+NR2A or NR1+NR2B) are transfected with constitutively active Src kinase to obtain high phosphorylation of the NR2 subunits. Activity of Src is monitored using phospho-specific Src antibodies (PY418 and PY529). NR2 subunits are precipitated from the cell-lysate with an NR2A or NR2B specific antibody and Src induced phosphorylation is detected with phosphospecific antibodies or a generic phosphotyrosine antibody using SDS-PAGE. In a similar experiment PTPMEG is co-transfected with Src and should reduce either Src phosphorylation or NR2A or NR2B phosphorylation. Both events lead to reduced NMDA-R currents in the presence of PTPMEG.

Activation of intracellular Src kinase in HEK293 cell can be obtained by stimulating serum starved HEK293 cells with growth factors (EGF, PDGF) at appropriate concentrations. Src activation is monitored by phosphospecific Src antibodies. Growth factor stimulation of the stable cell-lines in the presence or absence of PTPMEG will show increased or decreased NMDA-R phosphorylation and activity, respectively.

Calcium Imaging. The effect of a modulating compound upon NMDA-R is investigated by analysis of calcium flux through the channels upon activation or inactivation of the NMDA-R.

Measurements are done in presence/absence of compounds in a stable cell line inducibly expressing NMDA-R subunits as described above by using a FLEX station/Flipper or Ca²⁺ Imaging (see Renard, S. et al. Eur. J. Physicology 366:319-328 (1999)). The Molecular Devices FLEX station is a scanning fluorimeter coupled with a fluid transfer system that allows the measurement of rapid, real time fluorescence changes in response to application of compounds. As the function of NMDA receptors depends critically upon their ability to act as calcium channels that flux Ca2+ upon activation, the FLEX station in combination with calcium indicator dyes is used to measure NMDA receptor activity. This allows investigation of roles of interacting proteins in the modulation of both the magnitude and kinetics of NMDA receptor mediated calcium influx and screening for compounds that are able to modulate the functional properties of NMDA receptors. Stable cell lines, e.g. HEK cells inducibly expressing NMDA-R subunits are advantageous as they provide a homogenous population of cells, particularly useful for high throughput measurements in multi-well plate formats, which integrate the fluorescence properties of a population rather than individual cells.

For profiling assays, primary hippocampal or cortical neurons are infected with either Sindbis or Lentivirus constructs expressing the wt PTPMEG, the csPTPMEG and a GFP control. NMDA or L-Glutamate induced currents are recorded selectively in presence/absence of compounds. In order to measure NMDA currents, the cells are clamped with the patch pipette and characteristic NMDA-R currents recorded at different membrane potentials (Köhr & Seeburg, J. Physiol (London) 492: 445-452, 1996).

Neuronal NMDA receptor function is measured using either electrophysiology or the FLEX station, i.e measuring Ca²⁺ influx. Measurements are done in presence/absence of compounds in a primary neuronal cell expressing NMDA-R subunits as described above by using a FLEX station/Flipper or Ca²⁺ Imaging (see Renard, S. et al. Eur. J. Physicology 366:319-328 (1999)). The FLEX station in combination with calcium indicator dyes is used to measure NMDA receptor activity. Similarly to the experiments in HEK293, it is expected to see a decrease in NMDA-R current in neurons infected with the wt PTPMEG virus. Compounds would restore NMDA-R function/activity by inhibiting PTPMEG. The PTPMEG (cs) dominant negative mutant serves as a control.

### Example 3

### Measuring Activity of Agents that Modulate NMDA-R Signaling

Expression and Purification of PTPMEG The DNA sequence encoding the C-terminal 326 residues of PTPMEG (phosphatase domain) was subcloned into the pET-17b vector (Novagen) between the Ndel and Xhol restriction sites. An N-terminal tag consisting of Asp-Ser-6×His was incorporated at the beginning of the protein sequence. Protein expression was performed in BL21(DE3) cells at 37° C. with a 4 hour 0.5 mM IPTG induction. The cells were harvested by centrifugation and stored at −80° C. until needed for protein purification. Protein purification consisted of cell lysis by sonication, immobilized metal affinity chromatography (IMAC) on a Ni²⁺-NTA column (Qiagen), followed by hydrophobic interaction chromatography (HIC) on a phenyl sepharose column (Amersham Pharmacia Biotech) and/or ion exchange chromatography (IEC) on a Q sepharose column (Amersham Pharmacia Biotech). The purified 38.3 kDa protein was buffer exchanged into 50 mM HEPES, pH 7.5 and stored at −80° C.

Assay Development

TR-Fret Assay

Material:

Phosphatase Buffer: 50 mm HEPES, pH 8; 1 mM DDT; 2 mM EDTA; 0.01% Brij solution; 10 mM MgCl₂. Detection Buffer: 25 mM Tris, pH 7.5, 45 uM sodium orthovanadate; 0.5 μl Eu PY20 Ab; 0.75 μl Streptavidin-APC per 5 ml of Detection Buffer. *Buffers can be stored at 4° Celsius. Corning 384-well, black assay plate 3710. Substrate: AGY 1336. Enzyme: PTPMEG. Sodium Orthovanadate. DMSO (HPLC grade).

Method:

The enzyme stock solution is made by adding 20 μl PTPMEG stock (at 90 nM) to 100 ml of phosphatase buffer. The substrate stock solution is made by adding 1 μL AGY-1336 (at 5 mM) to 500 ml of phosphatase buffer. The control inhibitor stock solution is made by adding 10 μl sodium orthovanadate (45 mM) to 10 ml phosphatase buffer. The detection reagent stock solution is made by adding 15 μl Eu-anti-phosphotryosine antibody+45 μL APC to 150 ml of detection buffer. This yields initial concentrations of: Enzyme: 18 pM; substrate: 10 nM; vanadate: 45 uM.

The reagents for the control wells are dispensed by the Biomek 2000 (B2K) and Biomek FX robots. The B2K dispenses controls into six assay plates. 12.5 μl of enzyme, 2.5 μl of DMSO, and 10 μl of buffer is placed into column 1 and 2, rows A through H. A substrate volume of 12.5 μl, 2.5 μl of DMSO, and 10 μl of buffer is placed into columns 1 and 2, rows I through P. Column 23, row A through P will contain 5.0 μl of orthovanadate solution. Column 24 is left empty.

For the enzyme activity assay, 2.5 μl of compound, 12.5 μl of enzyme, and 10 μl of substrate (separated by air gaps) are added to columns 3 through 24 by the Biomek FX in a single dispense. After the dispense, the tips are washed with DMSO and water for re-use between each quadrant. Once the assay plates are set up, they are incubated at 27° C. for 45 minutes. Then 20 μl of detection buffer is added to stop the reaction and to allow the Europium antibody (Eu-Ab) and streptavidin-APC to bind to the substrate.

The plates are then placed in the plate reader, an Analyst HT. Excitation light at 360 nm is used to excite the Europium antibody with an emission at 620 nm. Fluorescence resonance energy transfer (FRET) from Eu-Ab to APC will only occur when they are in close proximity. Therefore, when an APC emission is observed at 665 nm the enzyme has been inhibited from removing the phosphate group from the substrate. The FRET assay is time-resolved (TR), where there is a delay between excitation light and collection of emission signals. This reduces the amount of stray light created by short-lived fluorescing molecules. The Analyst HT measures APC and Europium emission signals and calculates the ratio between the two intensities. Typical intensities for the Europium is ˜2000 and APC is ˜600.

The following compounds were tested by FRET against the listed phosphatases, and the IC50 were obtained. It can be seen that the inhibitors are selective for PTP-MEG.

| | | PTPMEG | CD45 | PTPIB | PTPB | T Cell | YOP51 |
|---|---|---|---|---|---|---|---|
| Object ID | Structure | IC50 (uM) | IC50 (uM) | IC50 (uM) | IC50 (uM) | IC50 (uM) | IC50 (uM) |
| AGY- 0041763 | 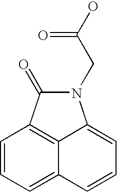 | 0.56 | >500 | N.D | >500 | >500 | N.D. |
| AGY- 0046293 | 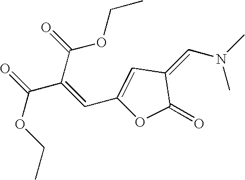 | 0.32 | 89.96 | 41.14 | 23.22 | 6715.24 | 4.18 |

### Example 4

### Animal Models for Validation of PTPMEG Inhibitors

Compounds that inhibit PTPMEG activity in both biochemical and functional assays are evaluated for potential antipsychotic activity in two animal models for schizophrenia; the amphetamine-induced hyperactivity model and the prepulse inhibition of acoustic startle model.

Amphetamine-induced hyperactivity model. The open field test chamber consists of a simple squared enclosure with infrared beams. The enclosure is configured to split the open field into a center and periphery zone. The total distance covered is used as an index of activity and locomotion whereas time and activity spent in the center of the open field were used as index of anxiety.

Pretreatment with d-amphetamine (5 mg/kg, SC) significantly increases locomotion in mice as shown by an increase in total distance spent in the open field over a 60 min recording period. The locomotor stimulant effect is significantly attenuated by clozapine (a reference compound) at a dose of 4 mg/kg administered 30 minutes prior to amphetamine.

The test compound was administered icv at different doses (50 μM, 100 μM, and 200 μM) to test its effects at attenuating the amphetamine-induced hyperlocomotion. The open field also measured anxiety, defined by a decrease in the distance and number of rearing observed in the center of the open field as compared to the periphery.

Prepulse inhibition of acoustic startle (PPI) model. The acoustic startle measures an unconditioned reflex response to external auditory stimulation. PPI is a measurement of reduced startle response to auditory stimulation following the presentation of a weak auditory stimulus. This measurement of PPI has been used to assess deficiencies in sensory-motor gating.

Animals were placed individually into the startle enclosures and secured in the startle chamber for a total of 40 minute test time. During the first 10 minutes, animals were acclimated to the chamber with a background noise of 70 dB. Following the acclimation period, a 30-min test ensued that consists of 56 trials. Each trial started with a 50 millisecond null period, followed by a 20 millisecond pre-pulse white noise sound of 72, 74 or 78 dB with a 100 millisecond delay preceding the startle stimulus (SS). The SS was a 40 millisecond 120 dB white noise sound that was followed by a 290 millisecond record time of startle. The response in the startle chamber was measured every millisecond for 65 milliseconds after the onset of the SS, or in PPI alone trials of the pre-pulse. In no-stimulation trials, a baseline measure was taken to assess movement under no stimulation. Clozapine 4 mg/kg was used as a reference compound and is administered 30 min prior to testing. Each dose of the test compound was administered through ICV injection and animals were immediately placed into the test chamber.

Mice treated with clozapine 4 mg/kg have lower startle response than mice receiving a vehicle treatment. A test compound was tested for effect on the startle response. Mice treated with clozapine also have significantly higher PPI than mice treated with vehicle, a profile well known for this atypical antipsychotic drug.

1. A method for identifying a modulator of N-methyl-D-aspartate receptor (NMDA-R) signaling activity, comprising detecting the ability of an agent to modulate the phosphatase activity of PTPMEG on a substrate or to modulate the binding of the PTP to NMDA-R, thereby identifying the modulator,
wherein active PTPMEG decreases NMDA-R signaling activity.

2. The method according to claim 1, wherein said PTPMEG is capable of dephosphorylating a protein tyrosine kinase (PTK), which PTK phosphorylates NMDA-R.

3. The method according to claim 2, wherein said PTK is Src.

4. The method according to claim 2, wherein said PTK is Fyn.

5. The method of claim 1, wherein the PTPMEG is human.

6. The method of claim 1, wherein the modulator is identified by detecting its ability to modulate the phosphatase activity of the PTPMEG.

7. The method of claim 1, wherein the modulator is identified by detecting its ability to modulate the binding of the PTP to the NMDA-R.

8. The method according to claim 1, wherein the modulator is identified by detecting its ability to modulate the dephosphorylation of NMDA-R by PTPMEG.

9. A method for identifying an agent as a modulator of NMDA-R signaling, comprising:
(a) contacting
(i) the agent
(ii) PTPMEG and a protein tyrosine kinase (PTK) that phosphorylates NMDA-R; and
(iii) NMDA-R or a subunit thereof;
wherein either or both of (ii) and (iii) is substantially pure or recombinantly expressed;
(b) measuring the tyrosine phosphorylation level of the NMDA-R or subunit;
(c) comparing the NMDA-R tyrosine phosphorylation level in the presence of the agent with the NMDA-R tyrosine phosphorylation level in the absence of the agent,
wherein a difference in tyrosine phosphorylation levels identifies the agent as a modulator of NMDA-R signaling and wherein active PTPMEG decreases NMDA-R signaling activity.

10. The method of claim 9, wherein said NMDA-R and said PTPMEG exist in a PTPMEG/NMDA-R-containing protein complex.

11. The method of claim 9, wherein said agent enhances the ability of PTPMEG to dephosphorylate said PTK.

12. The method of claim 9, wherein said agent inhibits the ability of PTPMEG to dephosphorylate said PTK.

13. The method of claim 9, wherein said agent modulates binding of PTPMEG to NMDA-R.

14. The method of claim 13, wherein said agent promotes or enhances binding of PTPMEG to NMDA-R.

15. The method of claim 13, wherein said agent disrupts or inhibits binding of PTPMEG to NMDA-R.

16. A method for identifying an agent as a modulator of NMDA-R signaling, comprising:
(a) obtaining a cell culture coexpressing the NMDA-R and PTPMEG
(b) introducing an agent into a portion of the cells; thereby producing cells comprising the nucleic acid molecule;
(c) culturing the cells in (b);
(d) measuring the tyrosine phosphorylation level of NMDA-R in the cells in (c) and comparing the level with that of control cells into which the agent has not been introduced
wherein a difference in tyrosine phosphorylation levels identifies the agent as a modulator of NMDA-R signaling.

17. A method for treating a disease mediated by abnormal NMDA-R-signaling, comprising administering a modulator of a PTPMEG activity, thereby modulating the level of tyrosine phosphorylation of NMDA-R.

18. The method of claim 17, wherein the modulator modulates the ability of PTPMEG to directly or indirectly dephosphorylate NMDA-R.

19. The method of claim 17, wherein the modulator modulates the ability of PTPMEG to bind to NMDA-R.

20. The method of claim 17, wherein the disease is selected from the group consisting of ischemic stroke; head trauma or brain injury; Huntington's disease; Parkinson's disease; spinocerebellar degeneration; motor neuron diseases; epilepsy; neuropathic pain; chronic pain; alcohol tolerance; schizophrenia; Alzheimer's disease; dementia; psychosis; drug addiction; ethanol sensitivity, mild cognitive impairment; and depression.

21. The method of claim 17, wherein the modulator is a PTPMEG antagonist and affects the ability of a protein tyrosine kinase to phosphorylate NMDA-R.

22. A method for identifying a modulator of Src protein tyrosine kinase activity, comprising detecting the ability of an agent to modulate the phosphatase activity of PTPMEG on Src,
wherein active PTPMEG decreases Src activity.

23. A method for identifying a modulator of Fyn protein tyrosine kinase activity, comprising detecting the ability of an agent to modulate the phosphatase activity of PTPMEG on Fyn,
wherein active PTPMEG decreases Fyn activity.
